(19) United States
(12) Patent Application Publication
(10) Pub. No.: US 2022/0193351 A1
(43) Pub. Date: Jun. 23, 2022

(54) Nasal Inhaler Band

(71) Applicant: Philip Wayne Cressman, Waterloo (CA)

(72) Inventor: Philip Wayne Cressman, Waterloo (CA)

(21) Appl. No.: 17/227,341

(22) Filed: Apr. 11, 2021

Related U.S. Application Data
(63) Continuation-in-part of application No. 15/192,977, filed on Jun. 24, 2016, now abandoned.

(51) Int. Cl. A61M 15/00; A61K 9/70; A61K 9/00

(52) U.S. Cl. CPC A61M 15/0001; A61K 9/007; A61K 9/703

(57) ABSTRACT

An exemplary nasal inhaler band is fitted on the upper lip below the nasal openings of a user. The device provides minimal restriction of ambient airflow into the nostrils during inhalation, while also providing a flow of beneficial vapors into such airflow. A main body of the device has an outer layer adapted to hold vapor-releasing solids, liquids or gels. The outer layer is air-permeable and absorbent. The main body also has a base layer contacting the upper lip. The base layer is impermeable, so that volatile solids, liquids or gels do not come into contact with the user's skin. The main body is held in position in contact with the nose and upper lip by elastic straps extending back from ends of the main body to wrap around the user's ears.

This application is a continuation in part of application Ser. No. 15/192,977, the entire contents of which are incorporated herein by reference. Applicant claims full priority based on the prior application.

### BACKGROUND

### Field of the Invention

The present invention relates generally to devices for the introduction of vapors to nasal passages, and more specifically to devices held in front of nasal entrances to provide a flow of vapor into nasal passages during inhalation.

### Description of Related Art

The inhalation of vapors such as menthol, eucalyptol, etc. has been used for a long time in traditional and home remedy treatments around the world for alleviation of various symptoms related to the respiratory system, such as restricted nasal patency. Aromatherapy has popularized the effects of smelling a variety of essential oils or herbs to improve mood. Smell is detected by the olfactory glands which can detect very small levels of such herbs and send signals to the brain's limbic area. It has been more recently found that certain herbal substances such as mint and eucalyptus have an effect on certain receptors. In particular, Transient Receptor Potential cation channel subfamily M member 8 (TRPM8) receptors, and Transient Receptor Potential ion channel member 1 (TRPA1) receptors. These receptors require a higher concentration of active substances. The TRPM8 and TRPA1 receptors send signals directly to the cerebral cortex via the trigeminal nerve, and these signals have psychopharmacologic effects.

Traditionally, menthol, eucalyptol or various menthol/eucalyptol containing plant material is added into very hot water (in a bowl or other container), and the vapors coming off the surface inhaled. Menthol does not dissolve in water, but the hot water melts the menthol crystals which then float on top of the water and give off menthol and water vapor generated through the application of heat. This approach is cumbersome, involves hot water that could be dangerous to handle, needlessly exposes the eyes to menthol vapors, and only provides short-term exposure to menthol vapors. There is also significant potential for exposure to excessive concentrations of menthol vapors, known to cause irritation.

A number of menthol and eucalyptol containing products for vapor inhalation are currently marketed to provide relief against various symptoms related to the upper respiratory system.

Various cylindrical or tube-shaped devices are marketed, containing (within the tube) a substrate saturated with menthol and/or other chemicals or herbal compositions. A user inserts one end of the tube into a nostril and inhales. The active ingredient in such devices is typically dissolved in a liquid, which is impregnated on a substrate. Air flowing through and around the substrate picks up menthol vapors and/or other vapors. Heat is generally not applied to the substrate to assist in generating the menthol vapors. One disadvantage of this kind of device is that it only provides short-term exposure to an unknown and variable flow of menthol vapors. Also, contact with the nose may contaminate the container with bacteria or viruses.

Various other vaporizers, including hand held and tabletop units, are currently marketed. These vaporizers use menthol and other essential oils in the form of solids, oils, or pads saturated with liquids; and provide menthol and other vapors to nasal passages. These devices work on a similar principle to the cylindrical plastic inhalers, with some added moisture or humidification. Vaporizers are more convenient than inhalers, but are still bulky and only useful for short-term application of menthol vapors.

Various nasal strips or expanders are also marketed. Some strips fit on top of the nose and are impregnated with menthol. Their main drawback is that the site of vapor release is on top of the nose. Concentrated vapors may be released very close to the user's eyes, where they can be irritating. In addition, the limited size and position of this kind of nasal strip severely curtails the amount of menthol stored for vaporization and the amount of menthol vapors available for inhalation. The amount of menthol vapors generated and inhaled is sufficient to trigger the olfactory glands, so that these kinds of nasal strips are able to be promoted as aromatherapy devices. However, the vapor is not concentrated enough to activate TRPM8 receptors.

Mentholated creams can be rubbed onto the skin near nasal passages and release menthol vapors in the proximity of nasal openings. However, this approach can only provide low concentrations of menthol vapors (and/or other vapors) in the proximity of the nasal passages. Another disadvantage is that the creams can contaminate or stain hands, clothing, etc. These creams may in some cases also cause skin irritation.

Medical plasters are also currently sold, and may contain up to 5% menthol in a gel solution. Such plasters have been marketed for application to the skin as a treatment for bruises or strains, but not as nasal inhalers.

Vapor-releasing face masks are also presently marketed. These masks are essentially variations of surgical or dust masks which have been modified to hold menthol solids, oils or gels and provide menthol vapors to breathing passages during inhalation. These masks typically retain the traditional design originally intended to protect the entire area around the nose and mouth against dust or other particulates or irritants. The size and configuration of such masks makes them somewhat inconvenient to put on or remove. In addition, such masks must be removed every time the user needs to clear her nasal passages or perform everyday activities such as eating, drinking or talking. Moreover, when the user is resting or sleeping while wearing a mask, they may find it uncomfortable. Furthermore, over the course of time, saturation of the entire mask fabric with menthol vapors may lead to release of such vapors in the proximity of the eyes, causing eye irritation.

There are a number of nasal bandages on the market that hold absorbent material directly under and against nasal openings to absorb blood and other fluids that result from injury, illness or surgical procedures. However, the overall size, shape and thickness of such devices is such that nasal breathing of ambient air is severely limited.

For aromatic products such as menthol, it is not necessarily the fragrant smell that provides most benefit for users. Research has established that I-menthol and 1,8-Cineole are triggers (agonists) to the TRPM8 receptor (also known as the cold and menthol receptor) present in the human nasal vestibules and lungs. Stimulation of the TRPM8 receptors by molecules of menthol or 1,8-Cineole are known to result in an improvement in nasal patency. 1,8-Cineole not only is an agonist to TRPM8, but also acts as an antagonist to TRPA1.

Stimulation of TRPM8 receptors also relieves the symptoms of “air hunger” (or dyspnea). Air hunger is a subjective feeling of quasi-suffocation experienced by patients who may otherwise have normal blood oxygen levels (i.e., blood oxygen levels that do not warrant supplementary oxygen treatment). Dyspnea episodes often occur multiple times during a day, lasting for five to ten minutes each time.

Thus there is an unmet need in the market for a nasal inhalation device: (i) that can be worn for extended periods of time (including during sleep or while talking or eating); (ii) in which the menthol and/or other active ingredient(s) does not come into direct contact with a user's skin; (iii) that is placed as far away from the eyes as practicable; and (iv) which allows for control of the amount of vapor introduced over various time periods to the nasal passages. There is a further need for such devices to be lightweight, and compactly packaged for ease of storage and carriage. There is also a need for such a device to provide convenient refill options where practicable. Moreover, the devices should be easy to put on and take off.

### BRIEF SUMMARY

The present invention responds to the above-described unmet market needs by providing a nasal inhaler band. This device is fitted on the upper lip below the nasal openings of a user. The device provides minimal restriction of ambient airflow into the nostrils during inhalation, while also providing a flow of beneficial vapors into such airflow. The device can be worn for extended periods of time, including during sleep and activities such as talking and eating. If the nose needs to be cleared, the device can simply be temporarily moved down to the chin, giving the user access to blow their nose or clean it with paper tissue. The device can be quickly and easily put on or taken off when required, and can be stored in an airtight container when not in use. The device is constructed such that when it is worn, volatile solids, liquids or gels do not come into contact with the user's skin.

In a typical embodiment, the present invention includes a main body, which is shaped and sized to fit on the upper lip under entrances to the nostrils. The main body has an outer layer adapted to hold vapor-releasing solids, liquids or gels. The outer layer is air-permeable and absorbent. The main body also has a base layer contacting the upper lip. The base layer is impermeable, and a thickness of the base layer is typically not more than 5 millimeters (mm). The main body is held in position in contact with the nose and upper lip by elastic straps extending back from ends of the main body to wrap around the user's ears. The main body is elongated and made of flexible materials. A back surface of the base layer is thus able to conform to and closely fit on the curved surface of a user's upper lip. Further or alternatively, the main body may be curved to match the overall convex shape of a user's upper lip.

### BRIEF DESCRIPTION OF THE DRAWINGS

FIG. 1 is a side view of a head of a user wearing a nasal inhaler band in accordance with an embodiment of the present invention.

FIG. 2 is an enlarged front view of the user wearing the inhaler band of FIG. 1, but only showing parts in the vicinity of the user's nostrils and lips.

FIG. 3 is a schematic, enlarged cross-sectional view of a main body of the inhaler band of FIG. 2, taken along line III-Ill thereof.

FIG. 4 is similar to FIG. 3, but showing an alternative embodiment of the main body.

FIG. 5 is similar to FIG. 3, but showing another alternative embodiment of the main body.

FIG. 6 is similar to FIG. 3, but showing still another alternative embodiment of the main body.

FIG. 7 is a front perspective view of a body of a nasal inhaler band in accordance with an alternative embodiment of the present invention.

FIG. 8 is a front plan view of an outer layer of a nasal inhaler band in accordance with another alternative embodiment of the present invention.

FIG. 9 is a bottom perspective view of a main body of a nasal inhaler band in accordance with yet another alternative embodiment of the present invention.

FIG. 10 is essentially a bottom plan view of the main body of FIG. 9.

FIG. 11 is similar to FIG. 10, but showing an alternative embodiment wherein the main body is curved to match the overall convex shape of a user's upper lip.

### DETAILED DESCRIPTION OF EMBODIMENTS

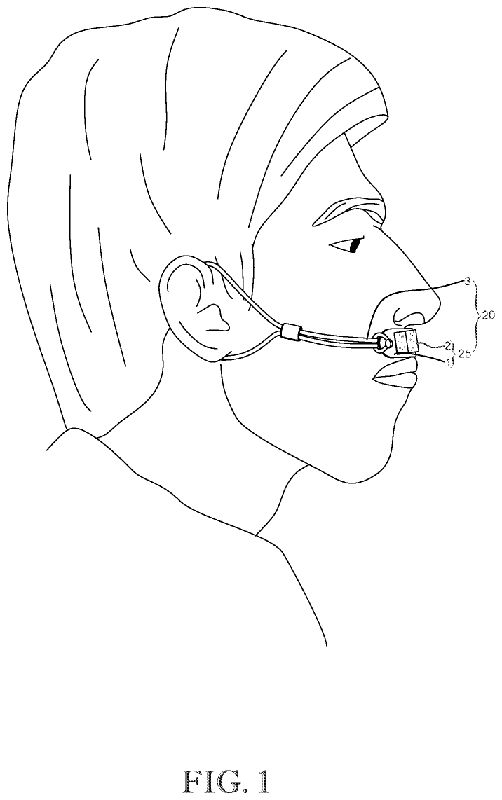

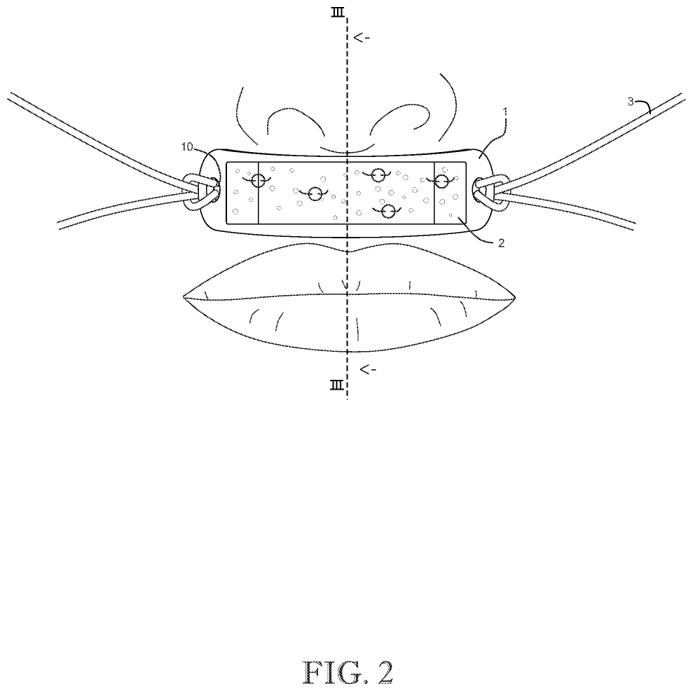

Referring to FIGS. 1 and 2, these show a nasal inhaler band (20) in accordance with an embodiment of the present invention. The inhaler band (20) comprises a main body (25), and elastic retaining straps or cords (3) for fitting around a user's ears. The main body (25) comprises an impermeable base or backside layer (1), and an outside air-permeable and absorbent layer (2). The base layer (1) is elongated in the form of a band (or strip), and is made of impermeable material. The outer layer (2) is also elongated in the form of a band (or strip), and is made of air-permeable and absorbent material. The outer layer (2) is adapted to hold and store vapor-releasing solids, liquids or gel substances. For example, the outer layer (2) may be impregnated with vapor-releasing solids, liquids or gel substances. Typically, the solids, liquids, or gel substances are volatile. The impregnation may be performed by the user, or may be performed in a factory during manufacture of the main body (25). At the time of impregnation, the vapor-releasing material is typically in the form of a solution or emulsion.

In this description, unless the context indicates otherwise, air-permeable is defined as permitting penetration and absorption of liquids or volatile vapors as well as air. Absorbent is defined as the ability of material to not only permit the penetration of air, liquids or volatile vapors, but also hold and contain some quantities of liquids, gels or solid forms of volatile substances. Fabric (woven or non-woven), felt and open cell foams are all considered to be air-permeable materials as well as being absorbent. Impermeable is defined as not permitting the penetration or absorption of liquids, air or vapors arising from volatile materials. Plastics, silicones and closed cell foams are all considered to be materials impermeable to liquids, air or vapors.

Each of the base layer (1) and outer layer (2) is made of flexible material, such as woven or non-woven fabric, plastics or silicones. Any such material may be in solid form, or in the form of open cell foam or closed cell foam, as required. For example, the base layer (1) is made of neoprene and the outer layer (2) can be made of terrycloth. Each of the base layer (1) and outer layer (2) can be considered to be in the shape or form of a tape, sheet, plate, board, band, or panel. In this embodiment, the base layer (1) and outer layer (2) are each approximately rectangular, are substantially parallel to each other, and are directly bonded to each other. Typically, the base layer (1) has a uniform thickness. Front ends of the elastic straps (3) are fastened to opposite ends of the base layer (1), by being passed through holes (10) in the opposite ends of the base layer (1) and then being fastened in place.

In alternative embodiments, the outer layer (2) comprises a plurality of sublayers stacked one on the other. Each sublayer is made of a selected air-permeable and absorbent material.

The Inhaler band (20) is positioned on a user's upper lip. This part of the human body is also known as the cutaneous upper lip. The main body (25) can bend or flex to follow the contours of the user's upper lip. Further or alternatively, the base layer (1) may be contoured or curved to match the overall convex shape of a human upper lip (see below). Typically, the base layer (1) is in contact with the columella (septum) of the nose and the alas (flares) of the two sides of the nose, with the inhaler band (20) centered on the philtrum (nasal crease). Thus the outer layer (2) is held directly below the user's nostrils, with substantially the full length of the base layer (1) in contact with the upper lip. In this way, the main body (25) is held in place by the elastic straps (3) extending back from the main body (25) to wrap around the ears of the user. Accordingly, unwanted upward movement of the main body (25) is prevented; and the solids, liquids or gel substances are prevented from contacting the user's skin.

With the inhaler band (20) in this position, the base layer (1) is slightly curved but not folded. In particular, the elongated base layer (1) snugly fits on the user's upper lip at the philtrum and at opposite left and right sides of the philtrum. Accordingly, blockage of air pathways below the nostrils, especially air pathways in the vicinity of a leftmost underside of the left nostril and a rightmost underside of the right nostril, is avoided or at least minimized. The main body (25) is thus shaped, sized, configured and positioned to prevent or at least minimize any obstruction of airflow into and out of the user's nostrils when the user breathes ambient air. Furthermore, the elastic straps (3) and the flexing and close fitting of the main body (25) on the user's upper lip cooperatively and resiliently retain the main body (25) in a position whereby any blockage or interruption of airflow to and from the user's nostrils is restricted to only inmost portions of entrances to the nostrils adjacent and corresponding in size to a thickness of the main body (25) along a horizontal length of the main body (25) below the nostrils.

FIG. 2 shows a width of a front face of the outer layer (2) is less than a width of a front face of the base layer (1), and a height of the front face of the outer layer (2) is less than a height of the front face of the base layer (1). Accordingly, an area of the front face of the outer layer (2) is less than an area of the front face of the base layer (1). With this configuration, a center of the front face of the outer layer (2) coincides with a center of the front face of the base layer (1), with the outer layer (2) positioned entirely within the boundaries of the base layer (1) and aligned with the base layer (1).

FIG. 2 also shows a decorative pattern printed on the outer layer (2). This helps the user to visibly distinguish the outer layer (2) from the base layer (1), so that the user fits the main body (25) onto his upper lip with the correct orientation. Alternatively, different solid colors, writing or other markings can be employed to clearly identify the two different sides of the main body (25).

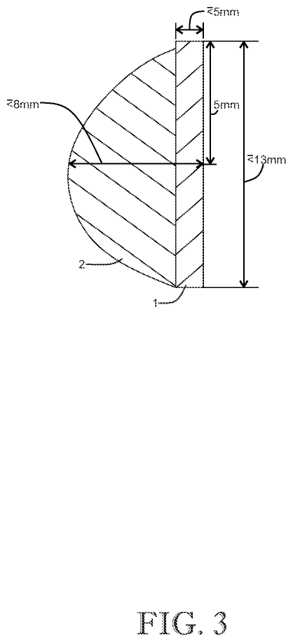

As shown in FIG. 3, in one embodiment, the outer layer (2) is in the form of a bulge. A thickness of the base layer (1), as measured from a front thereof to a back thereof, is uniform. In the illustration, the thickness of the base layer (1) is less than or equal to 5 mm. An overall thickness of the main body (25), as measured from a front thereof to a back thereof, is greatest at approximately a middle portion of the outer layer (2) located approximately halfway between a top of the outer layer (2) and a bottom of the outer layer (2). A thickness of the outer layer (2) gradually decreases from the middle portion thereof to each of top and bottom extremities thereof. In one embodiment, the bulge of the outer layer (2) is uniform along an entire horizontal length of the outer layer (2). That is, a transverse cross-section of the outer layer (2) taken anywhere along the length thereof would be the same as the cross-section of the outer layer (2) seen in FIG. 3. In this embodiment, the greatest thickness of the main body (25) is less than or equal to 8 mm.

In other embodiments, the bulge of the outer layer (2) is uniform along a majority of the horizontal length of the outer layer (2) except for opposite endmost portions of the outer layer (2). At these endmost portions, the outer layer (2) is thinner than at the majority portion.

It is known that the philtrum of an average person is in the range of approximately 11-13 mm in females and approximately 13-15 mm in males. In FIG. 3, the height of the base layer (1) is less than or equal to 13 mm, and the height of the outer layer (2) is approximately 10 mm.

The above-described dimensions are provided to avoid restricting airflow into and out from the user's nose during inhalation and exhalation. In addition, the sheet-like configuration and thinness of the main body (25) together with appropriate tension of the elastic straps (3) help prevent unwanted rolling up of the main body (25). Thereby, restriction of breathing airflow, and contact of the nose with volatile substances, are both avoided.

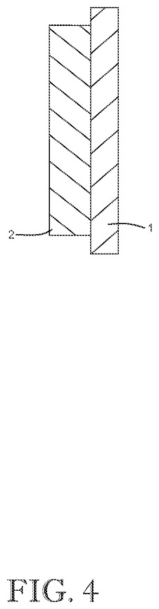

As shown in FIG. 4, in an alternative embodiment, a cross-section of the outer layer (2) is rectangular. In this embodiment, the rectangular configuration of the outer layer (2) is uniform along an entire horizontal length of the outer layer (2). The greatest thickness of the main body (25) is less than or equal to 8 mm. For example, the thickness of the main body (25) is approximately 6 mm. A thickness of the base layer (1), as measured from a front thereof to a back thereof, is less than or equal to 5 mm.

In one example, when the base layer (1) is made of neoprene and the outer layer (2) is made of terrycloth, the thickness of the base layer is approximately 4 mm, and the thickness of the outer layer(2) is approximately 1 mm to 1.5 mm.

In other embodiments, the rectangular configuration of the outer layer (2) is uniform along a majority of the horizontal length of the outer layer (2), except for opposite endmost portions of the outer layer (2). At these endmost portions, the outer layer (2) is thinner than at the majority portion.

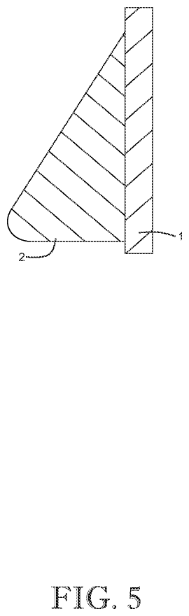

As shown in FIG. 5, in another alternative embodiment, the outer layer (2) becomes gradually thicker from a top extremity thereof to a bottom extremity thereof. That is, a thickness of the outer layer (2) is greatest at a bottom extremity thereof, and smallest at a top extremity thereof. In other words, a cross-section of the outer layer (2) is substantially triangular. In one embodiment, the configuration of the outer layer (2) is uniform along an entire horizontal length of the outer layer (2). In this embodiment, a greatest thickness of the main body (25) is less than or equal to 8 mm. A thickness of the base layer (1), as measured from a front thereof to a back thereof, is less than or equal to 5 mm.

In other embodiments, the triangular configuration of the outer layer (2) is uniform along a majority of the horizontal length of the outer layer (2), except for opposite endmost portions of the outer layer (2). At these endmost portions, the outer layer (2) is thinner than at the majority portion.

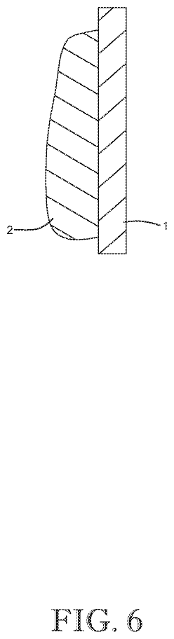

As shown in FIG. 6, in still another alternative embodiment, the outer layer (2) is in the form of a slightly bulged or contoured block. A thickness of the main body (25), as measured from a front thereof to a back thereof, is greatest at approximately a lower portion thereof. A thickness of the outer layer (2) gradually and slightly decreases from a point on a lower portion thereof where the thickness of the main body (25) is greatest to a top end thereof. The thickness of the outer layer (2) gradually and slightly decreases from the point on the lower portion thereof where the thickness of the main body (25) is greatest to a bottom end thereof. In one embodiment, the slightly bulged block configuration of the outer layer (2) is uniform along an entire horizontal length of the outer layer (2). In this embodiment, the greatest thickness of the main body (25) is less than or equal to 8 mm. A thickness of the base layer (1), as measured from a front thereof to a back thereof, is less than or equal to 5 mm.

In other embodiments, the slightly bulged block configuration of the outer layer (2) is uniform along a majority of the horizontal length of the outer layer (2), except for opposite endmost portions of the outer layer (2). At these endmost portions, the outer layer (2) is thinner than at the majority portion.

The various shapes and contours of the outer layer (2) as shown in FIGS. 3 and 5-6 and described above also help to visually distinguish the outer layer (2) from the generally thin and flat base layer (1). Thereby, the user can easily identify the two different sides of the main body (25).

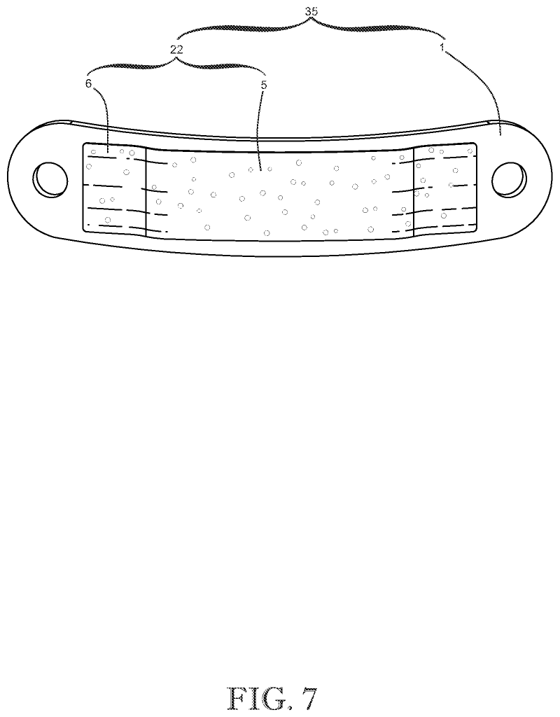

Referring to FIG. 7, in an alternative embodiment, a main body (35) of a nasal inhaler band comprises the base layer (1) and an outer layer (22). The outer layer (22) comprises a central pocket portion (5), and a pair of fixation portions (6) at opposite ends of the pocket portion (5) respectively. The pocket portion (5) is spaced from the base layer (1), and the fixation portions (6) are attached to the base layer (1). The base layer (1) and the pocket portion (5) cooperatively define a pocket. In the illustrated embodiment, the pocket has openings at a top and bottom thereof. In other embodiments, the bottom of the pocket may be sealed, with the only opening being at the top of the pocket. A matching air-permeable container (not shown) containing a vapor-releasing material (e.g., a therapeutic substance) can be inserted into and fittingly held in the pocket. The air-permeable container may be made of, or comprise, fabric. The container may be in the form of a cartridge, with each of multiple cartridges serving as a refill when needed. The outer layer (22) is made of air-permeable and absorbent material.

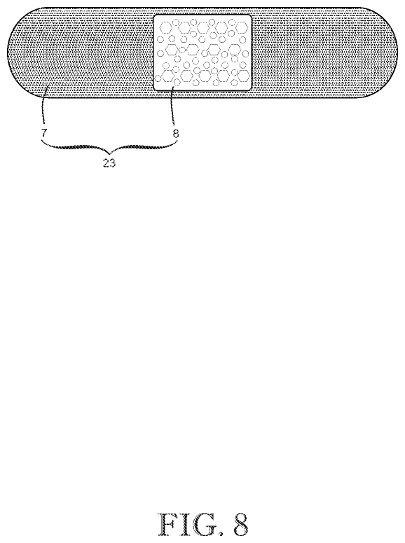

In another alternative embodiment shown in FIG. 8, an outer layer (23) of a main body of a nasal inhaler band comprises an air-permeable adhesive strip (7), and an air-permeable and absorbent vapor-releasing patch or pad (8) attached to a middle of a front face of the adhesive strip (7). The adhesive strip (7) can be an air-permeable adhesive plaster for example. The adhesive strip (7) has an adhesive back face, which is adhesively attached to a front face of the base layer (1) (not shown). The patch (8) can be impregnated with vapor-releasing solids, liquids or gel substances. The impregnation may be performed by the user, or may be performed in a factory during manufacture of the patch (8)

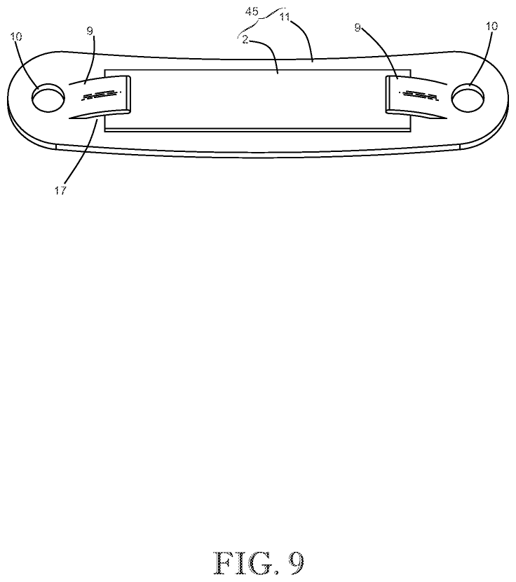

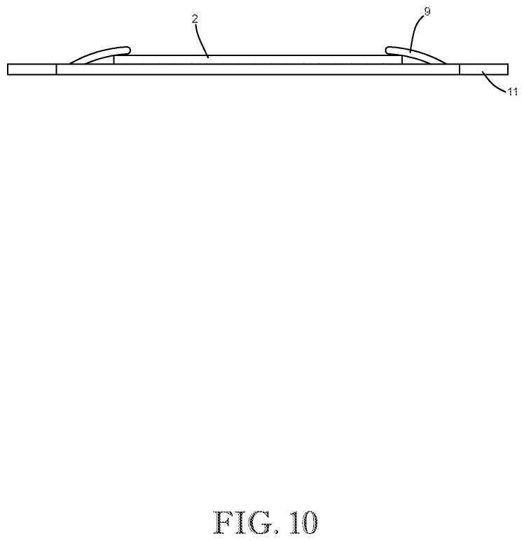

Referring to FIGS. 9 and 10, a main body (45) of a nasal inhaler band in accordance with yet another alternative embodiment of the present invention comprises a base layer (11) and the outer layer (2). The base layer (11) is typically made of flexible plastic or silicone material. The base layer (11) includes two molded tabs (or prongs) (9) at opposite ends thereof. The tabs (9) are located adjacent to inner ends of the holes (10) respectively. The tabs (9) extend obliquely outward from a body of the base layer (11) toward each other, thereby defining a pair of sockets (17). Thus when the base layer (11) is flexed, the sockets (17) can be opened slightly more to facilitate a user inserting the opposite ends of the outer layer (2) into the two sockets (17) and under the two tabs (9). Then when the (11) is released and recovers to its normal unflexed shape, the tabs (9) grip the outer layer (2), and the outer layer (2) is resiliently and firmly held in place by the base layer (11).

In other embodiments, the tabs (9) may be shaped such that the outer layer (2) may be installed and held in the sockets (17) without having to flex the base layer (11). Furthermore, the tabs (9) may be made of flexible material. In such case, the outer layer (2) can be installed and held in the sockets (17) without having to flex the base layer (11). The outer layer (2) can have a uniform thickness of approximately 6 mm, for example. Referring to FIG. 10, the absorbent layer (2) can have a thickness equal to or less than the thickness of the base layer (11).

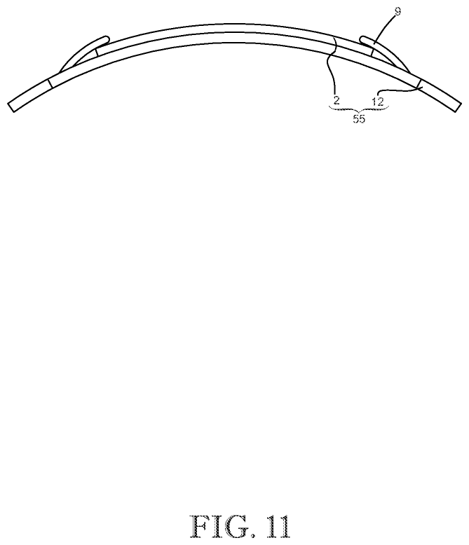

Referring to FIG. 11, a main body (55) of a nasal inhaler band in accordance with another embodiment of the present invention comprises a base layer (12) and the outer layer (2). The base layer (12) is curved, so that a concave side of the base layer (12) matches an overall convex curvature of a user's upper lip.

Any vapor-emitting TRPM8 agonist ligands or mixture of agonist ligands (suitable for human use) can be used as the active ingredient(s) for the inhaler band (20). For example, crystals, powder, flakes, particles, wax, liquids, pellets, blocks, powders, gels, films, sachets, and the like may be used. These substances and materials can be contained in a suitable carrier medium or vehicle, such as a solution, a suspension, a dispersion, an emulsion, a gel, liquids impregnated in a patch(es), pad(s) or sponge(s), crystals dissolved in eucalyptus oil or in other oil(s), liquids with solvent/gelling properties, or the like. For example, such media or carriers may be suitable for storing solid or liquid TRPM8 agonist ligands and for releasing vapors of such stored TRPM8 agonist ligands. Moreover, the TRPM8 agonist ligands may be releasably contained in microcapsules, an absorbent material, a cell, an adhesive, an emollient-containing composition, a solid support, a nanophase particulate structure, or the like.

Typical TRPM8 agonist ligands used are: menthol, cineole, geraniol, and any other similar volatile substances known to activate TRPM8 receptors.

For example, menthol is a volatile substance. Menthol crystals have a melting point of about 41-44 degrees Centigrade (deg. C.), and they sublimate even at room temperature. The rate of evaporation or 10 sublimation increases dramatically with temperature. The vapor pressure of menthol crystals at 20 deg. C. (room temperature) is 0.083 millimeters mercury (mm Hg). In contrast, when menthol crystals are heated to 34 deg. C. (a typical radiant temperature according to the present invention, achieved due to heat from the skin and exhaled air), the vapor pressure is 0.2385626 mm Hg. 1,8-Cineole, the principal ingredient of eucalyptol, has an even higher vapor pressure.

In the present invention, the nasal inhaler band is fitted on a user's upper lip just below the nasal openings. Thus the inhaler band can absorb heat from both the skin of the upper lip and air exhaled from the nostrils. Such absorbed heat raises the temperature of the inhaler band and the vapor-releasing material contained therein. This in turn generates a higher release rate of the vapor-releasing material, such as menthol or other TRPM8 agonist ligands. It may be assumed that the temperature of the vapor-releasing material approaches the temperature of the human body when the inhaler band is worn by a user. In such case, this may enable an approximate calculation of the vapor pressure of the TRPM8 agonist ligand at that temperature. Therefor at the time of manufacture of the inhaler band, a rough or even an accurate calculation of the quantity of TRPM8 agonist ligand to be preloaded in the inhaler band can be made. Accordingly, rough or even accurate control of the amount of vapor introduced over various time periods to the nasal passages can be achieved.

The nasal inhaler band of the present invention is lightweight and can be packaged in a sealed plastic bag or airtight container for ease of storage and carriage. Storage in an airtight container at temperatures below 35 deg. C. is expected to give a minimum of a two-year shelf life for the inhaler band.

While the foregoing written description of exemplary and preferred embodiments enable one of ordinary skill in the art to make and use the present invention, those of ordinary skill will understand and appreciate the existence of variations, combinations and equivalents of the specific embodiments, systems, methods and examples herein. Accordingly, the present invention should not be limited by the described embodiments, systems, methods and examples herein, and should include all embodiments, systems and methods within the scope and spirit of the claims herein.

1. A nasal inhaler band for releasing vapors to a user's nasal openings and allowing inhalation of the vapors along with ambient atmosphere, the nasal inhaler band comprising;
a main body comprising;
a base layer for applying to the user's upper lip, sized to fit flush on the user's upper lip below the user's nostrils, and in contact with the user's comella and both nasal wings at the nasal crease, the base layer being in the form of an elongated sheet and being made of flexible impermeable material; and an outer layer parallel to and attached to the base layer, the outer layer being in the form of a sheet and comprising air-permeable and absorbent material for storing a vapor releasing substance and allowing vapor produced by the vapor releasing substance to exit the outer layer in the vicinity of the user's nasal openings and wherein each of the base layer and the outer layer are elongated to extend horizontally beyond the user's nasal wings; and
wherein the width of the base layer is larger than the width of the outer layer; and
wherein the outer layer is attached to the base layer leaving an exposure of the base layer below the point of contact between the base layer and the user's comella and nasal wings; and
wherein the main body is fitted with a pair of elastic retaining straps attached to opposite ends of the main body respectively and configured for fitting around the user's ears.

2. (canceled)

3. The nasal inhaler band of claim 1, wherein the base layer is uniform in thickness as taken along an entire width and horizontal length of the base layer, and a variable thickness of the outer layer as measured at any point across the width is uniform along at least a majority of a horizontal length of the outer layer.

4. The nasal inhaler band of claim 3, wherein the maximum thickness of the outer layer taken along at least a majority of the horizontal length of the outer layer is the same thickness as or less than the maximum thickness of the base layer.

5. The nasal inhaler band of claim 3, wherein the maximum thickness measured along at least a majority of the horizontal length of the outer layer is in the range of between 2 mm and 6 mm, and the thickness of the base layer is in the range of between 2 mm and 5 mm.

6. (canceled)

7. The nasal inhaler band of claim 1, wherein a cross-section of the base layer, showing thickness and width taken along a front thereof to a back thereof, is substantially rectangular with a width larger than the width of a attached outer layer; and wherein a cross section of taken along at least a majority of the horizontal length of the outer layer, taken along a front thereof to a back thereof is, using geometric terms for non-classical shapes, a closed curve incorporating one or more straight lines, or a rectangle; and
wherein a cross section taken along at least a majority of the horizontal length of the main body of combined outer and base layers, as taken along a front thereof to a back thereof, is never in the shape of a rectangle but always a composite of two or more geometric shapes such as an irregular octagon or a closed curve consisting of one curved line connected at both ends at the side of a rectangular shape.

8. The nasal inhaler band of claim 1, wherein a material of the base layer is selected from the group consisting of plastic, silicone or closed cell foam; and a material of the outer layer is selected from the group consisting of absorbent woven fabric, non-woven fabric, felt, or open cell foam.

9. The nasal inhaler band of claim 1, wherein the outer layer comprises a plurality of sublayers stacked one on the other. wherein a material of each of the sublayers is selected from a group consisting of woven fabric, non-woven fabric, felt, or open cell foam

10. (canceled)

11. (canceled)

12. The nasal inhaler band of claim 1, wherein each of the base layer and the outer layer are elongated in length to extend beyond both sides of nasal wings and the base layer is held in contact with the user's comella and underside of the user's nasal wings at the nasal crease when retained by elastic retaining straps.

13. The nasal inhaler band of claim 1, wherein each of opposite end portions of the outer layer are attached to the base layer, a main central portion of the outer layer is spaced from the base layer, the main central portion of the outer layer and the base layer cooperatively define a horizontal pocket, having an opening at top, located under the user's nose and the bottom of the pocket may be sealed and the pocket is configured to fittingly and removably hold a matching cartridge, an air permeable container, containing vapor releasing substance that can be inserted by the end user.

14. The nasal inhaler band of claim 13, further comprising the matching cartridge.

15. The nasal inhaler band of claim 1, wherein the outer layers consist of a removable adhesive strip and an air-permeable and absorbent patch that is dimensionally smaller than the size of the adhesive strip in both width and length attached to a middle of a front face of said strip, the patch constituting the outermost layer of the outer layer of the inhaler band when attached; and
wherein the adhesive strip is removably adhered to the base layer of said device.

16. The nasal inhaler band of claim 1, wherein the main body is elongated and substantially straight when not applied to a user, the base layer consisting of flexible plastic or silicone material and includes flexible tabs at opposite ends thereof, the tabs extend obliquely outward from a body of the base layer toward each other, and the tabs grip the outer layer such that the outer layer is resiliently and firmly held in place against the base layer.

17. The nasal inhaler band of claim 1, the base layer consisting of flexible plastic or silicone material and wherein the main body is elongated and curved when not applied to a user; the base layer is curved so that a concave side of the base layer matches an overall convex curvature of a user's upper lip; the base layer includes flexible tabs at opposite ends thereof; the tabs extend obliquely outward from a body of the base layer toward each other; and the tabs grip the outer layer such that the outer layer is resiliently and firmly held in place in a middle of a front face of the base layer.

18. (canceled)

19. (canceled)

20. The nasal inhaler band of claim 1, comprising a pair of elastic retaining straps attached to opposite ends of the main body respectively and configured for fitting around the user's ears; wherein when the nasal inhaler band is worn by the user, the retaining straps apply inward and upward tension, and the flexing and skin contact fitting of the impermeable base layer against the user's upper lip cooperatively and resiliently retain the nasal inhaler main body in a position holding the base layer in contact with the user's comella and nasal wings at the nasal crease whereby any blockage or interruption of airflow to and from the user's nostrils is restricted at only external portions of entrances to the nostrils corresponding in amount of restriction to a thickness of the base layer along the horizontal length of the nasal inhaler base layer positioned below the nostrils.

21. The nasal inhaler of claim 1 wherein the elastic ear loops are attached to the main body of this device utilizing through holes located at opposing ends of said device.

22. The nasal inhaler of claim 1 wherein the cross-sectional shape of the absorbent outer layer taken along the horizontal length of the main body has an exposed and shaped surface profile formed by the outer layer joining with the base layer proximate to the vertical upper edge of the base layer, upper edge related to the user's upper lip when fitted, and moves outwards from the base layer in a downward curve, away from the user's nose and then inward towards the base layer in either a curve or a right-angled change of direction inwards and joins at the lower edge of the base layer; and wherein this shape performs the function of providing the flow of ambient air into the user's nostrils during inhalation.

23. The nasal inhaler band of claim 1 wherein the cross sectional shape of the exterior surface of the absorbent outer layer taken along the horizontal length of the outer layer is formed by contact with the base layer proximate to the upper vertical edge of the base layer, as fitted to the user's upper lip, moving outwards from the base layer at a right angle for a distance range of between 2 mm and 6 mm then curving or making a right angle downwards and continuing downwards before turning back towards the base layer and contacting the lower edge of the base layer completing the shape and; wherein this shape is functional as variable thickness of the absorbent layer in combination with a junction with the base layer at a point below the top edge of the base layer which contacts the user's comella and nasal wings below the nasal entrances provides a clear pathway for ambient air to move into the user's nostrils during inhalation.
